(19) United States
(12) Patent Application Publication
(10) Pub. No.: US 2009/0325212 A1
(43) Pub. Date: Dec. 31, 2009

(54) DATA STANDARD FOR BIOMATERIALS

(75) Inventors: Chris Demetrios Karkanias, Sammamish, WA (US); Sonia Patricia Carlson, Kirkland, WA (US); Simon John Mercer, Issaquah, WA (US)

Correspondence Address: LEE & HAYES, PLLC, 601 W. RIVERSIDE AVENUE, SUITE 1400, SPOKANE, WA, 99201, US

(73) Assignee: MICROSOFT CORPORATION, Redmond, WA (US)

(21) Appl. No.: 12/163,478

(22) Filed: Jun. 27, 2008

(51) Int. Cl. C12Q 1/02; G08B 23/00

(52) U.S. Cl. 435/29; 340/573.1

(57) ABSTRACT

Provided are systems and/or methods that facilitate sensing, detecting, logging, or treatment of a condition or need of a living body using a genetically engineered organism and a data standard.

### BACKGROUND

There have been numerous improvements in electronics useful in the monitoring of body functions. For example, solid state electronics are used to monitor heart arrhythmia, respiratory rate and solutes/glucose. Healthcare personnel use all kinds of medical monitoring devices including blood pressure monitors, blood glucose meters, ear thermometers, body weight monitors, body fat analyzers and oximeters to measure the physiological indices of patients so as to keep track of their physical conditions.

Usually the devices record data indicative of the body function, for example heartbeat or cardiovascular system or cardio-respiratory system, and store the data in a memory device. The recorded data is then taken to a physician or technician for interpretation and comparison with reference data. In the example of a heart attack, the time lapse between the actual event that triggered the problem and the analysis of the recorded data may be too long to help the patient. It may be necessary to promptly dispense medicine or apply treatment to help overcome the problem detected and the time delay between the event and the analysis of the recorded data may prove to be too late.

In many of the above applications, however, monitoring is limited to short periods of time due to the necessity of professional assistance and by limitations associated with the monitoring devices and the sensors themselves. Monitoring human physiological data on an extended, real-time basis presents many advantages to scientific researchers, medical professionals and individuals with a high level of interest in their own physiological condition.

### SUMMARY

The following presents a simplified summary of the innovation in order to provide a basic understanding of some aspects described herein. This summary is not an extensive overview of the claimed subject matter. It is intended to neither identify key or critical elements of the claimed subject matter nor delineate the scope of the subject innovation. Its sole purpose is to present some concepts of the claimed subject matter in a simplified form as a prelude to the more detailed description that is presented later.

In order to effectively manage, monitor, and/or treat a condition in the body, the necessity of obtaining feedback about activities, properties, changes, and treatments associated with the body and/or condition as well as any problems that may be occurring. The subject innovation relates to systems and methods that have comprehensive and flexible logging capabilities. The logging capabilities facilitate the transfer of accurate and reliable physiological information from within a body to an external infrastructure that can monitor a condition, as well as also provide a customized treatment for the condition, in light of the unique properties of a specific body.

In accordance with one aspect of the claimed subject matter, a system that facilitates monitoring a condition of a living body. The system includes an engineered organism that produces a biomaterial containing encoded information. An interface component facilitates delivery of the biomaterial to an operation component that analyzes the biomaterial using a data standard. The data standard associating each of a set of stimuli with a corresponding each of a set of biomaterial subunit sequences.

In accordance with another aspect of the claimed subject matter, methods of logging events in a living body involve introducing an engineered organism into a living body, the engineered organism capable of encoding events in a biomaterial. The biomaterial is collected and using a data standard, encoded data is correlated with the event.

In accordance with another aspect of the claimed subject matter, an event logging system that obtains and records data concerning a living body involves the use of an engineered organism that produces a biomaterial containing encoded information in response to stimulus and a data standard for correlating each of a set of stimuli with a unique biomaterial sequence code.

The following description and the annexed drawings set forth in detail certain illustrative aspects of the claimed subject matter. These aspects are indicative, however, of but a few of the various ways in which the principles of the innovation may be employed and the claimed subject matter is intended to include all such aspects and their equivalents. Other advantages and novel features of the claimed subject matter will become apparent from the following detailed description of the innovation when considered in conjunction with the drawings.

### BRIEF DESCRIPTION OF THE DRAWINGS

FIG. 1 illustrates a block diagram of an exemplary system that facilitates sensing, detecting, logging, or treatment of a condition of a living body using an engineered organism.

FIG. 2 illustrates an exemplary engineered organism and various functions.

FIG. 3 illustrates a block diagram of an exemplary system that facilitates sensing, detecting, logging, or treatment of a condition of a living body using an engineered organism, a controller and data store.

FIG. 4 illustrates a block diagram of an exemplary system that facilitates sensing, detecting, logging, or treatment of a condition of a living body using an engineered organism.

FIG. 5 illustrates a block diagram of an exemplary system that facilitates sensing, detecting, logging, or treatment of a condition of a living body using an engineered organism and an analysis component.

FIG. 6 illustrates a block diagram of an exemplary system that facilitates sensing, detecting, logging, or treatment of a condition of a living body using an engineered organism and artificial intelligence.

FIG. 7 illustrates a block diagram of an exemplary system that facilitates sensing, detecting, logging, or treatment of a condition of a living body using an engineered organism and alarms, logging, and/or querying.

### DETAILED DESCRIPTION

The claimed subject matter is described with reference to the drawings, wherein like reference numerals are used to refer to like elements throughout. In the following description, for purposes of explanation, numerous specific details are set forth in order to provide a thorough understanding of the subject innovation. It may be evident, however, that the claimed subject matter may be practiced without these specific details. In other instances, well-known structures and devices are shown in block diagram form in order to facilitate describing the subject innovation.

The systems described herein contain engineered organisms capable of producing biomaterials adhering to a data standard, an external infrastructure capable of interpreting the biomaterial and/or directing stimuli to the engineered organism. Organisms are engineered to produce biomaterials that encode information using a data standard. Knowledge of the data standard permits the interpretation of information encoded within the biomaterials by an infrastructure external to the organism. The external infrastructure is capable of reading the biomaterial to decode information therein and directing activity of the engineered organism using a type of stimulus.

Using the data standard, engineered organisms log physiological information concerning a living body in which the engineered organism resides. The logging capabilities facilitate the transfer of the physiological information from within the living body to an external infrastructure capable of reading an event log produced by the engineered organism. The external infrastructure can monitor a condition of the living body and/or provide a customized treatment for the condition. In some instances, the external infrastructure can induce action, such as treatment, relating to the condition by the engineered organism.

Generally speaking, the data standard is desirably stable and not self modifying and has a grammar associated therewith. The data standard requires use and selection of repeating subunits within a biomaterial. When using amino acid sequences or nucleotide sequences as the biomaterials representing data, typically in the form of a peptide, protein, or DNA sequence, the requirements of a data standard are satisfied. Using biomaterials to represent data, allows for storage of data at the molecular level. Biomaterials contain repeating subunits, can be produced by an engineered organism, and are capable of benign or beneficial existence within a living body.

According to the data standard, arbitrary data is represented in protein or DNA form. The data standard for biomaterials allows for a connection between an organism and an external infrastructure. The connection enables monitoring of a living body containing the engineered organism (and/or its environment) as well as effecting a desired activity by the engineered organism (and/or its environment). The data standard allows for sequential encoding of logfile entries into a polymeric biomaterial using an engineered coding scheme.

For example, the data standard encodes binary (in the case of a peptide/protein, coding involves use of two amino acids while in the case of DNA, coding involves use of two nucleic acids, both analogous to the use of “1” and “0” in computer code), although any encoding length can be employed. In other words, two different polymeric repeating units represent “1” and “0” in conventional computer binary code. Base-4, base-8, and base-16 coding systems can be alternatively employed.

The engineered organism constitutes biological hardware for writing data in the form of a produced biomaterial (either an amino acid or DNA sequence). Expressing binary code enables the expression of ASCII code, Baudot code, Braille code, ISO 8859-1 character code, Unicode, Huffman code, arithmetic code, EBCDIC code, UTF-8 code, UTF-16 code, or UTF-32 code, which then allows the expression of any standard within the biomaterial produced by the engineered organism. When the data standard employs binary code, two-bit binary codes, three-bit binary codes, four-bit binary codes, five-bit binary codes, six-bit binary codes, seven-bit binary codes, eight-bit binary codes, 16-bit binary codes, or 32-bit binary codes may be utilized. The data standard thus allows for programming of and event logging by engineered organisms. That is, certain functions of the engineered organisms can be created and/or controlled to further a therapeutic, diagnostic, or monitoring purpose.

Specific amino acid sequences or specific nucleotide sequences constitute code and are associated with discrete information. The data standard is the skeleton key required to interpret information represented by specific amino acid sequences or specific nucleotide sequences. The data standard is retained in an external infrastructure that is subsequently employed to read the biomaterial sequences. The data standard thus contains a list of elements and a corresponding biomaterial sequence for each element. The elements include events that may or may not occur with the living body and/or concerning the engineered organism. An exemplary list of elements includes the presence, absence, or threshold levels of a material produced by or part of a living body (such as insulin, adrenalin, a specific antibody, testosterone, estrogen, fatty acids, etc.); the presence, absence or threshold levels of a material ingested (and optionally derived from the ingested material) by a living body (such as glucose, a pharmaceutical compound, cholesterol, ethyl alcohol, nicotine, fatty acids, etc.); threshold properties (pH, temperature, electrolytes, heart beat rate, etc. above/below a predetermined level); production of a compound by the engineered organism; signaling received by or produced by the engineered organism; and the like. The foregoing list of elements is exemplary and many other elements can be represented by a biomaterial sequence, but for brevity, all are note listed.

Once the data standard is established, information can be written by producing a biomaterial, such as an amino acid sequence or DNA sequence, containing a sequence of bits (each bit represented by a polymeric subunit). The information can be read by determining the sequence of the biomaterial, such as by obtaining the specific amino acid sequence or DNA sequence.

Using the data standard, the engineered organism logs events into a log file. That is, information representing an event is written or captured by producing a biomaterial having a sequence associated with the information. Event logging using the data standard provides a standard methodology to record desired events.

Returning to the system, an engineered organism such as a genetically modified microorganism is provided to a living body for monitoring and/or therapeutic purposes, such that the engineered organism responds to a defined repertoire of stimuli or event elements. For each stimulus or event in the repertoire, the response of the engineered organism is a discrete state change specific to the type of stimulus applied including the production of log entry into a log file.

Living bodies are organisms that advantageously host an engineered organism. An example of a living body is a human being, although additional examples include other non-human mammals such as pets and livestock.

An engineered organism is capable of existing in a living body for a desired period of time and capable of at least one of monitoring, sensing, signaling, and/or treating various conditions of the living body. Often times, an organism which is genetically transformed into an engineered organism is capable of naturally existing in the living body, such as naturally occurring E. coli. Examples of engineered organisms include genetically engineered or otherwise modified biological material (for example, an organism may have a property or characteristic added, modified, or removed to provide the engineered organism). In other words, the modified biological material constituting the engineered organism can be artificially adapted for therapeutic, monitoring, or logging purposes. Engineered organisms also include the progeny of engineered organisms that are not themselves engineered. The engineered organism is in possession of a characteristic that facilitates evasion from or withstanding the effects of the immune system and/or is adapted to live inside or on the living body.

General examples of engineered organisms include bacteria, viruses, fungi, algae, plants, ciliates, parasites, pathogens, microorganisms, synthetic material such as hybridomas, and the like. A general example of bacteria specifically includes friendly or unharmful bacteria. It is noted that engineered organisms specifically include parasites, parasitic bacteria, and pathogenic bacteria since the positive effects and advantages outweigh the negative effects and disadvantages, especially when the negative effects and disadvantages of parasites, parasitic bacteria, or pathogenic bacteria can be reduced or minimized.

Genetically engineered refers to any recombinant method used to create an engineered organism that expresses state change, such as producing an event log embodied by a biomaterial or produces a signal of interest. Methods and vectors for genetically engineering organisms are known. Examples of genetic engineering techniques include expression vectors, targeted homologous recombination and gene activation, and transactivation by engineered transcription factors.

Specific examples of engineered organisms include genetically engineered bacteria of the genus Escherichia such as E. coli, bacteria of the genus Staphylococcus such as S. epidermis and S. aureus, those of the genus Arthrobacter such as A. agilis, A. citreus, A. globiformis, A. leuteus, A. simplex, those of the genus Azotobacter such as A. chroococcum, A. paspali, A. brasiliencise, and A. lipoferum, those of the genus Bacteroides such as B. lipolyticum and B. succinogenes, those of the genus Brevibacterium such as B. lipolyticum and B. stationis, those of the genus Kurtha such as K. zopfil, those of the genus Myrothecium such as M. verrucaria, those of the genus Pseudomonas such as P. calcis, P. dentrificans, P. flourescens, and P. glathei, those of the genus Phanerochaete such as P. chrysosporium, those of the genus Streptomyces such as S. fradiae, S. cellulosae, and S. griseoflavus, those of the genus Eubacterium, bacteria of the genus Bifidobacterium such as B. adolescentis, B. bifidum, B. animalis, B. thermophilum, B. breve, B. longum, B. infantis and B. lactis, Clostridium, Enterococcus, Fusobacterium, Peptostreptococcus, Ruminococcus, bacteria of the genus Lactobacillus such as L. acidophilus, L. bifidum, L. bulgaris, L. casei, L. bifidolongum, L. fermentum, L. brevis, L. cellobiosus, L. crispatus, L. curvatus, L. GG, L. gasseri, L. johnsonii, L. plantarum, L. salivarus, L. reuteri, L. delbrueckii, L. rhamnosusthose, those from the genus Saccharomyces (yeasts) such as S. cerevisiae, bacteria of the genus Actinomycetes, bacteria of the genus Azotobacter, bacteria of the genus Bacillus such as B. brevis, B. macerans, B. pumilus, B. polymyxa, B. subtilis, B. anthracis and B. cereus, bacteria of the species Bacteroides, bacteria of the genus Bordetella such as B. pertussis, bacteria of the genus Borrelia such as B. burgdorferi, bacteria of the genus Corynebacterium such as C. xerosis and C. hoffmanni, bacteria of the genus Campylobacter such as C. jejuni, those of the genus Candida such as C. albicans, Cyanobacteria, bacteria of the genus Deinococcus such as D. radiodurans, bacteria of the genus Enterococcus, bacteria of the genus Micrococcus, bacteria of the genus Streptococcus such as S. thermophilous and S. faecium, those of the genus Oceanobacillus, those of the genus Synechocystis, those of the genus Pseudomonas, those of the genus Helicobacter, those of the genus Mycobacterium, those of the genus Arabidopsis, those of the genus Thermotoga, and the like.

An additional source of cell lines and other biological material for use as engineered organisms or as starting material to make engineered organisms may be found in ATCC Cell Lines and Hybridomas, Yeast, Mycology and Botany, and Protists: Algae and Protozoa, and others available from American Type Culture Collection (Manassas, Va.), all of which are herein incorporated by reference.

Parasites include human parasites and non-human parasites such as animal parasites. Parasites are adept at penetrating into and residing in living body hosts or on a human body. Owing to the natural affinity of parasites to specific areas of a living body, parasitic engineered organisms can be advantageous in some situations where a specific organ or tissue of the living body is targeted for treatment, detection, or investigation. In instances where the treatment includes oncological treatments of a specific organ, using parasitic engineered organisms is particularly advantageous. When using a parasitic engineered organism, it is in some instances helpful to mitigate the pathogenic affects of the parasite.

Specific examples of parasitic engineered organisms include Balantinium coli (which typically burrows into the intestinal mucosa), trypanosoma brucei (a hemoflagellate), those of the genus Plasmodium (intracellular parasites of liver and red blood cells), nematodes such as Ascaris lumbricoides (roundworm which typically invades the gastrointestinal tract and lungs), pinworms such as Enterobius vermicularis (gastrointestinal tract, colon, fingertips), whipworms such as Trichuris trichiuria (gastrointestinal tract), flukes or trematodes such as Fasciola hepatica and schistosomes (liver and gallbladder), tapeworms or cestodes such as those from the genus Taenia (gastrointestinal tract), hookworms, heart worms, roundworms, Trichomonas vaginalis (vagina, urethra, epididymis, and prostate), spirochetes such as saprospira, cristispira, treponema (blood and lymphatic systems), lice (head, body, and pubic), mites, scabies, those from the genus Entamoeba such as E. histolytica, those of the genus Cryptosporidium, those of the genus Schistosoma such as S. mansoni, S. haematobium, and S. japonicum, those of the genus Borrelia such as B. burgdorferi (brain), and the like.

The engineered organism can be such that indefinite presence in the living body is possible, or that temporary presence is possible (for example, if the engineered organism is ingested as a part of food, the engineered organism may pass through the digestive tract and exit the living body in the feces). An advantage associated with the engineered organism is that the duration or presence in the living body is controllable. In other words, techniques exist for removing the engineered organism from the living body when desired.

The engineered organism can be removed or deactivated by a suitable dosage of a disinfecting compound such as an antibiotic. The disinfecting compound either destroys, deactivates, or otherwise causes the engineered organism to leave the living body without deleteriously affecting the living body. Examples of the disinfecting compound include chemical biocides, disinfectants, antibiotics, and chemotherapeutic agents. Alternatively, the engineered organism can be engineered to self destruct or initiate apoptosis under controllable conditions, such as when a self destruct signal is received or the conditions in the living body activate a self destruct mechanism.

Engineered organisms can be made using known techniques. For example, recombinant DNA techniques may be employed to make a genetically engineered bacterium having a desired function. Recombinant DNA is typically in the form of a vector. The vector may for example be a plasmid, cosmid, phage, or artificial chromosome. Vectors frequently include one or more selectable markers to enable selection of cells transformed (or transfected: the terms are used interchangeably in this specification) with them and, preferably, to enable selection of cells harboring vectors incorporating heterologous DNA. Appropriate “start” and “stop” signals are typically present. Additionally, if the vector is intended for expression, sufficient regulatory sequences to drive expression are present. Cloning vectors can be introduced into friendly bacteria, E. coli, or another suitable host which facilitates their manipulation.

Nucleic acid sequences for making the genetically engineered organism may be prepared by any convenient method involving coupling together successive nucleotides, and/or ligating oligo- and/or poly-nucleotides, including cell-free in vitro processes, but recombinant DNA technology forms the method of choice. Ultimately, nucleic acid sequences can be introduced into cells of the organism by any suitable means.

Nucleic acid sequences can be introduced into cells of the organism by transformation using known techniques. Alternatively, nucleic acid sequences can be introduced into cells of the organism by electroporation and vacuum filtration procedures. Alternatively, foreign DNA can be introduced directly into cells of the organism using an electrical discharge apparatus. Any other suitable method that provides for the stable incorporation of the nucleic acid sequence within the nuclear DNA or mitochondrial DNA of host cells can be employed.

The engineered organism can be introduced into the living body by natural or artificial techniques including oral ingestion (such as in food), nasal, rectal, vaginal, injection, topical or transdermal, and the like. One advantage associated with using parasitic engineered organisms is that such organisms tend to naturally enter a living body and target specific tissues or organs. The mode of administration is not critical.

The repertoire of stimuli or event elements include, for example a library of micro RNAs, peptides, small molecules (such as aspirin), or physical stimuli such as light of a specific wavelength. As mentioned above, the repertoire of stimuli or event elements also includes the presence, absence, or threshold levels of a material produced by or part of a living body; the presence, absence or threshold levels of a material ingested by a living body; threshold physiological properties; production of a compound by the engineered organism; signaling received by or produced by the engineered organism; and the like.

The repertoire of stimuli can be physiologically inert (so that there are minimal deleterious side effects on the engineered organism or deleterious effects on the living body, if any), or the repertoire of stimuli can be physiologically active. In the case of molecular stimulus, they may degrade over time—and in some instances the degradation can be a desirable property, by limiting the range or duration of the stimulus. In the case of some types of molecular stimulus, the engineered organism can have an additional modification to include a transmembrane pore or receptor which can accommodate the stimulus, triggering a state change within the engineered organism.

The repertoire of stimuli or events can also include conditions or components of the living body. Examples of conditions or components of the living body include vitamin levels/production, mineral levels, amino acid levels/production/reduction, fatty acid levels/production, hormone levels/production, insulin levels/production, hemoglobin levels/production, glucose levels, alcohol levels, infections including viral infections and bacterial infections, parasitic infections, genetic disorders, cancer (colon cancer, liver cancer, bone cancer, brain cancer, stomach cancer, prostate cancer, breast cancer, ovarian cancer, cervical cancer, lung cancer, etc.), cancer produced substances such as various toxins, the undesirable presence of toxins, abnormal physical states, allergen levels (such as to insect bites/stings, pest/snake bites, food allergies, and the like), drug metabolite levels, immune response compounds (such as IgG, IgM, IgA, IgD, or IgE, etc.) levels/production, melanin levels/production, and the like.

The repertoire of stimuli or events can also include chemical signals, radiation or light signals, electromagnetic signals, fluorescence, a detectable physical change in the living body (color change) and the like. A chemical signal can be generated by secretion of a detectable chemical, peptide, or protein (the detectable chemical or protein can be present in a blood sample, feces sample, etc.).

The conditions are generally conditions of the living body, but also specifically include the conditions within local areas/tissues such as various conditions of the blood, plasma, digestive tract, feces, liver, lungs, lymphatic system, muscle tissue, bone marrow. Thus, generally speaking, a repertoire of stimuli or events include the presence, absence, or amount (including too high or too low) of a biologically necessary substance or a pathogen. Since conditions or components of the living body that can be sensed and recorded by the engineered organism, the conditions or components can be easily monitored by the engineered organism. The conditions or components of the living body that can be sensed and recorded by the engineered organism can be treated by the engineered organism or by a separate regimen of activity.

The data standard of biomaterials requires encoding in the engineered organism of each of the cellular states inducible by the repertoire or events of external stimuli. In some instances the data standard of biomaterials can optionally and additionally involve the encoding in the engineered organism of quantitative measures (for example, the ability to write an event log when a given threshold of specifically identified molecules are detected by the engineered organism). In some instances, the data standard of biomaterials can optionally and additionally involve encoding in the engineered organism an embedded error correction protocol (at least a checksum) to ensure the data are encoded unambiguously.

Taking the example of amino acids, 20 standard amino acids are used in nature (although synthetic non-natural variants—which increase the flexibility of the code because negative biological activity is of no or little concern). In one implementation, two amino acids can be used to create a binary encoding, which confers absolute flexibility in expressiveness at the expense of length. In another implementation, nineteen amino acids can be used in combination to encode each cellular state plus digits, with the twentieth acting as a spacer; assuming a triplet code (one of many choices), giving 6859 permutations. A subset of the permutations can be selected to avoid unintentional biological activity and undesirable structural properties such as unbalanced hydrophobicity/hydrophilicity and problematic conformations.

Referring to the receptor or pore possessed by the engineered organism, the nature of the active site and the nature of the repertoire of chemical stimuli designed to interact with the site can be defined by a second data standard. Such data standard can be useful when producing the engineered organisms, and the second data standard can be retained in the external infrastructure. In effect, the second data standard functions as an interface between the engineered organism and the repertoire of stimuli or event elements.

The state changes induced by the repertoire of stimuli or event elements can be permanent in the engineered organism (such as triggering cell death), or transient (such as inducing transcription of a gene). Some state changes can trigger natural behaviors the engineered organism already possesses (such as cell division or motility) while other state changes can trigger activation or suppression of an artificial construct inserted in the engineered organism, such as expression of a fluorescent protein present on an inserted vector, or activation of an event log.

In one aspect of the system described herein, each state change of the engineered organism includes an associated logfile entry such that when logfile activity is activated, the stimulus or event is entered into an event log. The logfile entry can be a chemical released inside the engineered organism (such as a microRNA, DNA, or amino acid sequence), which results in a characteristic signature appearing in an ordered logfile output. The logfile output embodied by a biomaterial is produced in accordance with the data standard for biomaterials and contains information relating to the biology, chemistry, physiology at a given point in time in the engineered organism and/or living body.

The biomaterial containing the logfile output typically is a polymer of at least two different subunits, and is often biologically inert, although in some instances the biomaterial containing the logfile output can trigger biological activity within the living body or by the engineered organism such as immune responses. However, if biological activity within the living body or by the engineered organism is desired, a discrete instruction output from the engineered organism is typically made, the discrete instruction output separate from the biomaterial containing the logfile output. The biomaterial containing the logfile output is capable of forming polymers of unrestricted length, such as DNA or amino acid sequences. Polymers having an endedness feature (identifiable and distinguishable ends) are particularly useful because an order of events logged therein may be inferred from the encoding with an understanding of starting and ending ends. For example, DNA has a 5-prime and 3-prime end. As another example, peptides and proteins have an N-terminal end containing free amino group and a C-terminal end containing free carboxyl group. Typical examples of biomaterials containing the logfile output include peptides, proteins, and DNA. In some instances, RNA, long-chain fatty acids, and carbohydrates may be useful as biomaterials.

Logfile writing is initiated by one of the repertoire of stimuli or events referred to above, and writing can be terminated by another stimulus or event from the same repertoire. Yet another stimulus can signal the engineered organism to release the logfile into the surrounding medium where the logfile output can be recovered for reading. That is, a stimulus or event can induce the engineered organism to express or produce the biomaterial containing the logfile and into the living body so that the biomaterial can be collected, such as by collecting blood, urine, saliva, feces, mucus, hair, or other bodily discharge and subject to analysis.

The engineered organism is capable of producing the logfile by excreting or expressing a biomaterial containing the logfile. The biomaterial can be excreted or expressed by the engineered organism as a single entity, or the biomaterial can be a constituent of a substance (e.g., a DNA sequence in a virus, encapsulated by a fluid, fat, or carbohydrate, and the like). The engineered organism releases the biomaterial containing the log file at either a predetermined time or upon response to a given stimulus or event.

Once the biomaterial is collected from the living body, standard techniques for isolating the biomaterial are employed. After isolating the biomaterial, standard techniques for determining the polymer sequence are employed. For nucleotide sequences, for example, any of chain-termination methods, Maxam-Gilbert sequencing, chain-termination methods, dye-terminator sequencing methods, high-throughput sequencing technologies, parallelized sequencing, methods involving DNA polymerase, and the like may be employed. For nucleotide sequences, for example, any of mass spectrometry, Edman degradation, and the like may be employed.

In one implementation, logfile reading can involve serial digestion of a protein biomaterial (chemically) to determine the sequence of polymer subunits. If DNA is used as the biomaterial, a spacer between events/signals can be designed that permits enzymatic restriction with the size of the resulting fragments being used to infer sequence, or labeled complementary nucleotides can be hybridized to perform the same function.

Another alternative implementation can involve the production of active logfiles. If the system is designed such that any logfile sequence can be generated with an appropriate sequencing of the repertoire of stimuli, then a subset of the possible stimulus sequences can produce logfile molecules with defined biological activities (if the logfile molecule was DNA or RNA, an appropriate set of stimuli can encode a viral payload, and trigger the cell to produce virus).

The treatment of conditions of the living body by the engineered organism using active logfiles in a controllable manner is a significant advantage associated with the subject innovation. Treating in a controllable manner means that the actual treatment of a condition is provided in response to a stimulus or signal, not an artificial indication. For example, the engineered organism may release a compound for the treatment of a condition when a predetermined stimulus or event is detected by the engineered organism. Alternatively, the engineered organism may release a compound for the treatment of a condition when a state is detected within the living body (e.g., a pH exceeding a threshold pH, a glucose level exceeding a threshold glucose level, the presence of toxin, etc.) through event logging, and logfile reading.

The capability of engineered organism to report a condition based on the biomaterial logfile permits communication of valuable information, often near real time information, from within the living body. The capability of engineered organism to report a condition based on the biomaterial logfile a condition permits communication of information that otherwise may not exist due to the absence of indicators readily detectable by casual human observation. Such information is valuable in that it is unbiased and free from subjective interpretation, thereby mitigating a human beings ability to misrepresent a condition of the body. Relaying information is one or two way in that it alternatively or additionally includes the capability to signal the engineered organism external to living body. The capability of signaling the engineered organism permits the engineered organism to act, such as treat a condition, based on a stimulus from outside the living body (such as after diagnosis). The capability to report a condition or signal the engineered organism can be controlled external to living body.

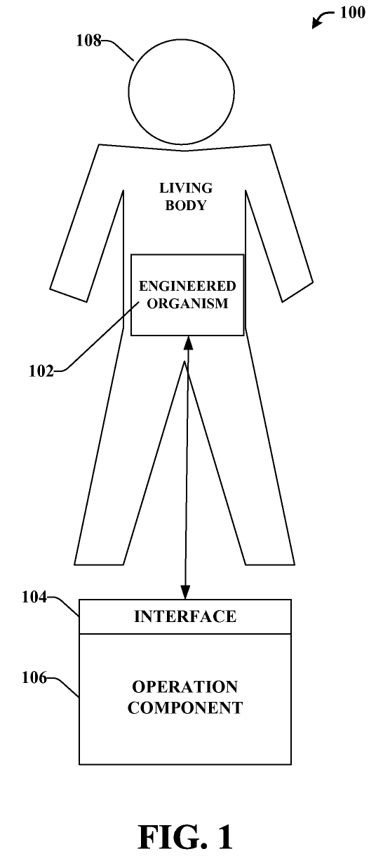

Turning now to the drawings, FIG. 1 illustrates a system 100 that facilitates event logging within a living body using a data standard. An engineered organism 102 can provides event logging and monitoring related to one or more particular conditions, wherein the condition can be associated with the living body, with the engineered organism and/or external factors affecting the living body. The engineered organism 102 can be within or on the living body 108 such that various conditions can be monitored and logged, optionally in real-time, to provide accurate and unbiased data associated therewith. An operation component 106 (or external infrastructure) can analyze the encoded data generated by the engineered organism 102 within the living body 108 via an interface 104 (discussed infra) using a data standard. The operation component 106 can provide a plurality of analysis based at least in part upon the received data related to a condition associated with the living body 108. The operation component 106 contains a data standard for the biomaterials produced by the engineered organism 102. More specifically, the data standard correlates a specific stimulus (events that cause logging) to specific subunit sequences of the polymeric biomaterial. Consequently, the operation component can read the encoded data generated by the engineered organism 102. The operation component 106 essentially converts the biomaterial output of the engineered organism 102 into readily understandable event log, which tracks activities and conditions in the living body 108. For example, the operation component 106 can monitor a specific condition of the living body 108 based at least in part upon data generated by the engineered organism 102.

The engineered organism 102 can be placed within the living body to provide in situ measurements, wherein the operation component 106 can analyze such data to determine at least one condition of the living body. The engineered organism 102 can be placed, for instance, within the gastrointestinal tract, lungs, bladder, circulation system, muscle system, bone marrow, head, fatty tissue, or other internal organs. With the engineered organism 102 collecting and encoding data associated with the living body and creating a record of the encoded data, the operation component 106 can provide diagnoses, recommendations, interventions, or other manipulations based at least in part upon the decoded data generated by analyzing the encoded data using the data standard.

Furthermore, the system 100 can include any suitable and/or necessary interface component 104 (herein referred to as “interface 104”), which provides various adapters, collectors, conduits, connectors, channels, communication paths, receivers, purifiers, sequencers, etc. to integrate the operation component 106 into virtually any operating and/or database system(s). The interface 104 can receive encoded data or biomaterials containing the encoded data, wherein the data received can relate to the engineered organism 102; the living body 108; data associated with the living body; systems associated with the living body 108; collected data about a condition; any suitable data related to a living body condition, etc. In addition, the interface component 104 can provide various adapters, collectors, connectors, channels, communication paths, receivers, etc., that provide for interaction with the operation component 106. The results of the sensed information may be used to direct future analysis and/or symbiotic agent operation. For example, a change in chemistry detected by the symbiotic agent may result in a signal to further “probe” the living body for particular species representing the same or different condition that may be causing the change in chemistry.

The interface 104 may be used to send signals to the engineered organism 102. These signals enable the engineered organism 102 to function as an active monitor. For example, a non-human entity is detected in the vicinity of the engineered organism. This detection may be for example initiated by signals to the engineered organism 102. The characteristics of the signals may change over time and may be defined by operation component 106. The signal can cause the engineered organism to dynamically alter the chemistry thereby addressing the non-human entity.

It is to be appreciated that the system 100 can include a plurality of different engineered organisms 102 and the system 100 depicted is not to be limiting. For instance, the system 100 can include a plurality of engineered organisms 102 with a single interface 104; multiple engineered organisms 102 with multiple interfaces 104 and a plurality of operation components 106; etc. In other words, the system 100 can utilize any suitable number of engineered organism, interfaces, and/or sensor operation components to track, monitor, collect, and/or record, in situ data associated with the living body 108. Moreover, it is to be appreciated that the interface component 104 and the operation component 106 can be stand-alone components and not embedded in the living body environment.

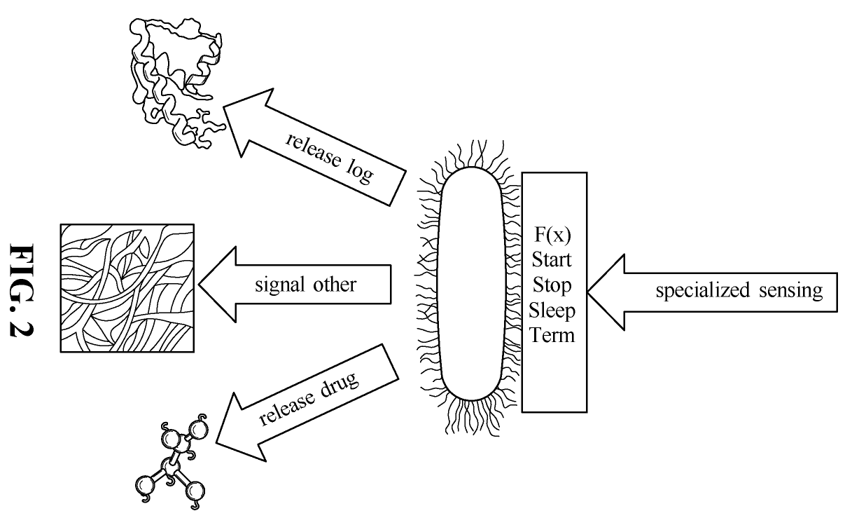

FIG. 2 illustrates an engineered organism, such as that described in FIG. 1, and associated functions. The specialized sensing includes a repertoire of stimuli that induce some type of response by the engineered organism. FIG. 2 illustrates three functions that engineered organism can implement in response to any given stimulus. The engineered organism can release a drug or some other compound useful for treating a condition in the living body. The engineered organism can signal another engineered organism, a cell within the living body, or an interface to an external infrastructure. The engineered organism can alternatively or additionally release a log written in a biomaterial that is subsequently decoded by an external infrastructure. The data standard can be used to define the specialized sensing, the signaling, and/or to read and write the event log.

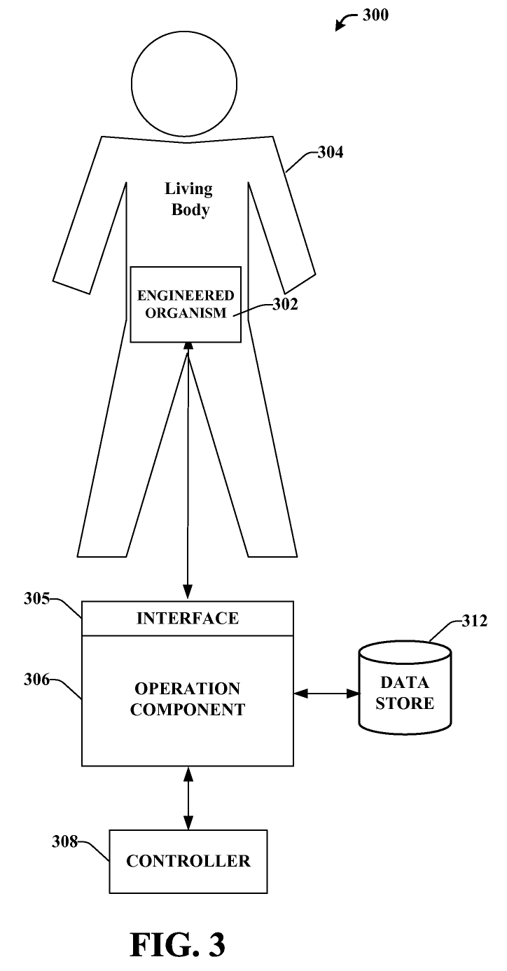

FIG. 3 illustrates a system 300 that facilitates detecting characteristics associated with a living body and recording the characteristics in an event log that utilizes an engineered organism capable of producing a biomaterial in accordance with a data standard. The event log is in the form of encoded data of the sequences of a biomaterial produced by the engineered organism 302. The system 300 includes a living body 304 that includes an engineered organism 302 that provides encoded data associated thereto via an interface 305. The engineered organism 302 can measure various conditions, sense various conditions, and log events associated with the living body 304 to provide control, diagnosis, improved safety, improved health quality, and/or improved operation. Furthermore, an operation component 306 can provide analysis, processing, etc. based at least in part upon a data standard and the encoded data generated by the engineered organism 302. It is to be appreciated that the engineered organism 302, the living body 304, and the operation component 306 can be different or substantially similar to the engineered organism 102, the living body 108, and the operation component 106 of FIG. 1, respectively. Although a single engineered organism 302 is illustrated in the system 300, it is to be appreciated and understood that the claimed subject matter is not so limited and any number of engineered organism can be utilized. The multiple engineered organisms may operate by exchanging data and/or analysis between each other or the operation component.

The in situ encoded data collected, detected, generated, signaled and/or monitored data related to the living body 304 can be analyzed and/or processed by the operation component 306. Using the data standard for biomaterials, operation component 306 translates the biomaterial sequence into useful information related to the living body 304. Furthermore, the operation component 306 can provide data manipulations based at least in part upon the data collected, generated, detected, and/or monitored by the engineered organism 302 within the living body 304. Moreover, the decoded data generated by the operation component 306 can be utilized by a controller 308 coupled to the operation component 306. In accordance with an aspect of the subject innovation, the controller 308 can be a programmable logic controller (PLC). While PLCs can be utilized within the system 300 as the controller 308, it is to be understood that any suitable automation controller can be employed in connection with the claimed subject matter. For example, any suitable microprocessor and/or microcontroller can be utilized within the system 300 as the controller 308 including a personal computer or single-board computer with suitable input-output circuitry. Moreover, it is to be appreciated that the controller 308 can contain software components and hardware components having inputs and/or outputs that can be utilized in connection with analyzing or identifying conditions of the living body.

The operation component 306 can utilize a data store 312, wherein the data store 312 can store various data related to the system 300. The data store 312 can provide storage for in situ encoded data generated by the engineered organism 302, decoded data generated by operation component 306 using the data standard, the configurations, conditions, health standards, control signal, system response, quality, disease/illness information, profiles, historic data, medical data, calibration data, security data, safety data, etc. The data store 312 can be, for example, either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable programmable ROM (EEPROM), or flash memory. Volatile memory can include random access memory (RAM), which acts as external cache memory. By way of illustration and not limitation, RAM is available in many forms such as static RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), Rambus direct RAM (RDRAM), direct Rambus dynamic RAM (DRDRAM), and Rambus dynamic RAM (RDRAM). The data store 312 of the subject systems and methods is intended to comprise, without being limited to, these and any other suitable types of memory. In addition, it is to be appreciated that the data store 312 can be a server, a database, a hard drive, and the like.

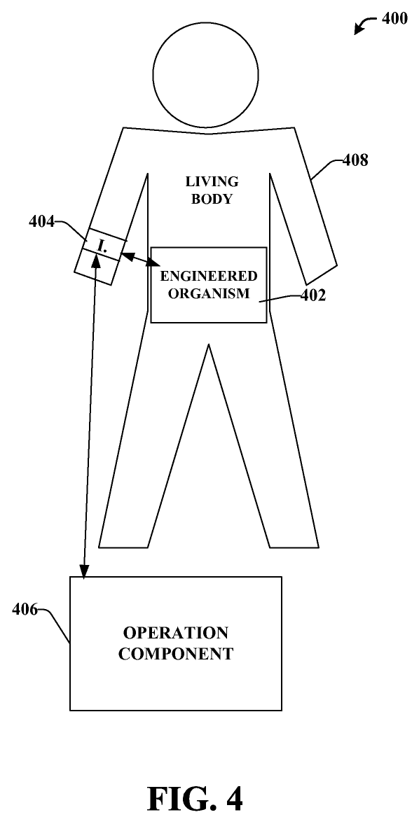

FIG. 4 illustrates a system 400 that facilitates implementing an engineered organism within a living body using an interface closely associated with a living body. An engineered organism 402 can provide sensing, event logging, and/or measurement related to one or more particular conditions, wherein the condition can be associated with the living body and/or external factors affecting the living body. Event logging is captured in encoded data embodied by a biomaterial produced by the engineered organism 402. The engineered organism 402 can be within or on the living body 408 such that various conditions can be sensed, measured, logged, and/or detected, optionally in real-time, to provide accurate and unbiased data associated therewith. An operation component 406 (or artificial processing center) can analyze the encoded data generated by the engineered organism 402 within the living body 408 via an interface 404 (discussed infra).

The system 400 can include any suitable and/or necessary interface component 404 (herein referred to as “interface 404”), which provides various adapters, collectors, connectors, conduits, channels, communication paths, receivers, transmitters, isolators, sequencers, etc. to integrate the operation component 406 with the engineered organism and into virtually any operating and/or database system(s). The interface 404 can receive encoded data, wherein the encoded data received can relate to the engineered organism 402; the living body 408; data associated with the living body; systems associated with the living body 408; collected data about a condition of the living body; any suitable data related to a living body condition, etc. In addition, the interface component 404 can provide various adapters, collectors, connectors, channels, communication paths, conduits, receivers, transmitters, isolators, etc., that provide for interaction with the operation component 406. While the interface 404 as shown is an arm band or watch, the interface may be positioned at any location of the living body most suitable to communicate with the engineered organism 402. For example, the interface 404 may be incorporated into a hat, glasses, a ring, shoes, an ankle strap, belt, or other clothing articles. The interface 404 may be positioned within the living body 408, such as within a pacemaker, a cochlear implant, artificial joint/limb, etc.

The interface 404 may be used to send signals to the engineered organism 402. These signals enable the engineered organism 402 to function as an active sensor. For example, a non-human entity such as a pathogen, may be detected in the vicinity of the engineered organism. The signal can cause the engineered organism to dynamically alter the chemistry in the living body thereby addressing the non-human entity.

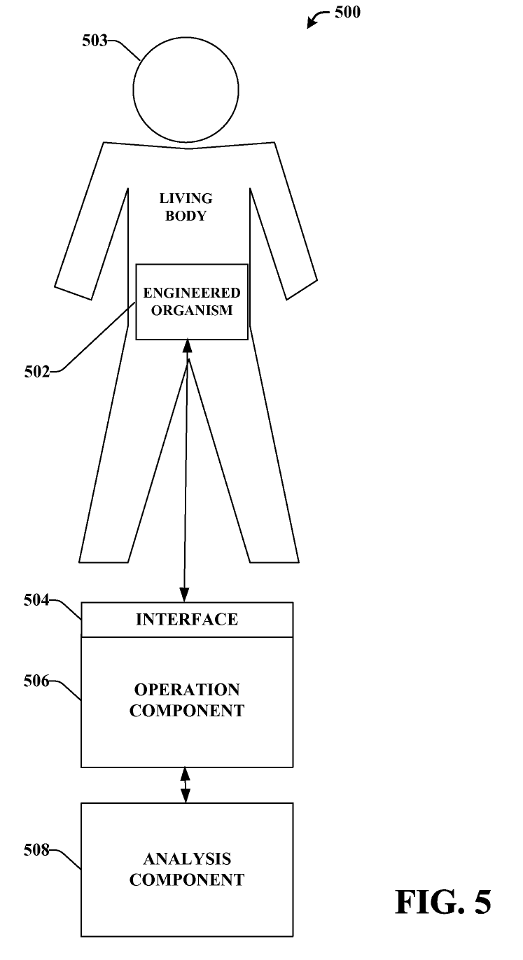

FIG. 5 illustrates a system 500 that facilitates detecting illness conditions within a living body. The system 500 can include an in situ engineered organism 502 that provides continuous sensing, monitoring, logging, and/or detection in a living body 503 includes an engineered organism 502. It is to be appreciated that the engineered organism 502, operation component 506, and the analysis component 508 can be substantially similar to previously described components. The analysis component 508 can analyze encoded data generated by the engineered organism 502 or decoded data generated by the operation component 506 in order to identify an illness or disease. For example, the engineered organism 502 can include a number of different receptors specific for various different maladies. The engineered organism 502 signals the operation component 506 via the interface 504 with encoded data that can be extrapolated to determine which receptor is occupied, and what the significance of the bound receptor is. The encoded data is decoded by the operation component 506 using the data standard. Although the interface 504 is shown directly coupled with the operation component 506, the interface 504 may be directly coupled with the living body 503, such as a device held within a wrist band, ankle band, clothing article, shoe, etc.

The analysis component 508 may contain a classifier. A classifier is a function that maps an input attribute vector, x=(x1, x2, x3, x4, xn), to a confidence that the input belongs to a class, that is, f(x)=confidence(class). Such classification can employ a probabilistic and/or statistical-based analysis and can further be influenced by a consideration of utilities or costs associated with correct and incorrect classification to prognose or infer an action that a user desires to be automatically performed. A support vector machine (SVM) is an example of a classifier that can be employed. The SVM operates by finding a hypersurface in the space of possible inputs, where the hypersurface splits the triggering criteria from the non-triggering events. Intuitively, this makes the classification correct for testing data that is near, but not identical to training data. Other model classification approaches include, e.g., naive Bayes, Bayesian networks, decision trees, neural networks, fuzzy logic models, and probabilistic classification models assuming different patterns of independence can be employed. Classification as used herein also is inclusive of statistical regression that is utilized to develop models of priority.

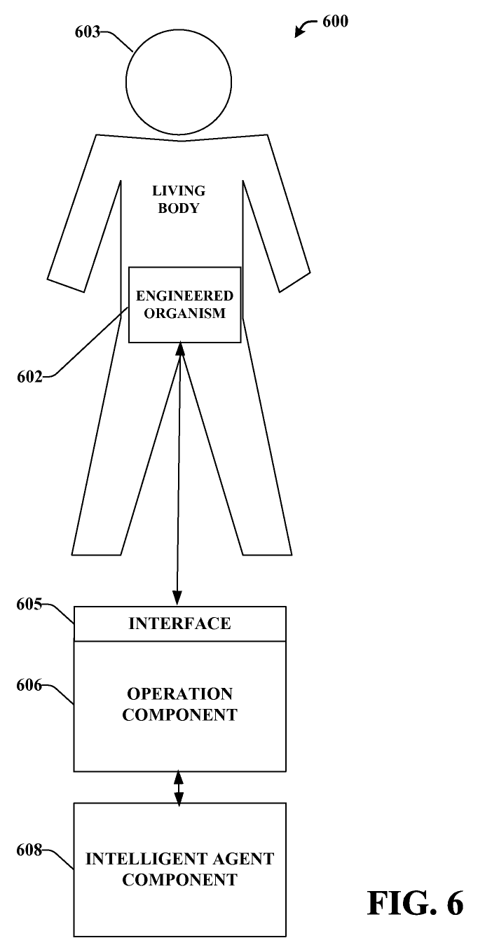

FIG. 6 illustrates a system 600 that facilitates monitoring a living body with engineered organism in real-time. The system 600 can include an engineered organism 602 that can be incorporated into a living body 603. An operation component 606 can provide decoded data using a data standard and data manipulations based at least in part upon the collected real-time encoded data, wherein such manipulations can provide treatment, diagnosis, prognosis, control, detection, of a condition of the living body 603.

The system 600 can include an intelligent agent component 608 that can be deployed on commercially available PLC's, PC's, SBC, etc. within or connected to the operation component 606. It is to be appreciated that the claimed subject matter can include multiple types of engineered organisms 602 and/or multiple intelligent agent components 608. Moreover, it is to be understood that the intelligent agent component 608 can be a stand-alone component or incorporated into the operation component 606. The intelligent agent component 608 can provide holonic system capabilities and the eventual transition to autonomous agents, which can respond to unexpected detections/data, the ability to dynamically respond to the unexpected detections/data. The intelligent agent component 608 can allow highly distributed diagnostics to sense an unfavorable condition and prescribe a superior countermeasure.

The intelligent agent component 608 can utilize a suite of simulation, prototyping, and/or deployment tools in accordance with the subject innovation. By deploying the intelligent agent component 608, unprecedented intervention can be provided as well as consequent overall health benefits. The system 600 can significantly enhance the quality, diagnostic reliability, health, and efficacy of living bodies. Furthermore, the real-time process information can be utilized for closed-loop feedback control, adaptive process model development, predictive treatment, and other intervention applications.

The system 600 can enable the living body to function during illness, deficiency, or disease. Moreover, the intelligent agent component 608 can adhere to published agent-to-agent communication protocols (e.g., Foundation for Intelligent Physical Agents (FIPA)) and provide local intervention and decision-making along with more overall health goals.

It is to be understood that the intelligent agent component 608 can provide for reasoning about or infer states of the system, environment, and/or user from a set of observations as captured via events and/or data. Inference can be employed to identify a specific context or action, or can generate a probability distribution over states, for example. The inference can be probabilistic—that is, the computation of a probability distribution over states of interest based on a consideration of data and events. Inference can also refer to techniques employed for composing higher-level events from a set of events and/or data. Such inference results in the construction of new events or actions from a set of observed events and/or stored event data, whether or not the events are correlated in close temporal proximity, and whether the events and data come from one or several event and data sources. Various classification (explicitly and/or implicitly trained) schemes and/or systems (e.g., support vector machines, neural networks, expert systems, Bayesian belief networks, fuzzy logic, data fusion engines . . . ) can be employed in connection with performing automatic and/or inferred action in connection with the claimed subject matter.

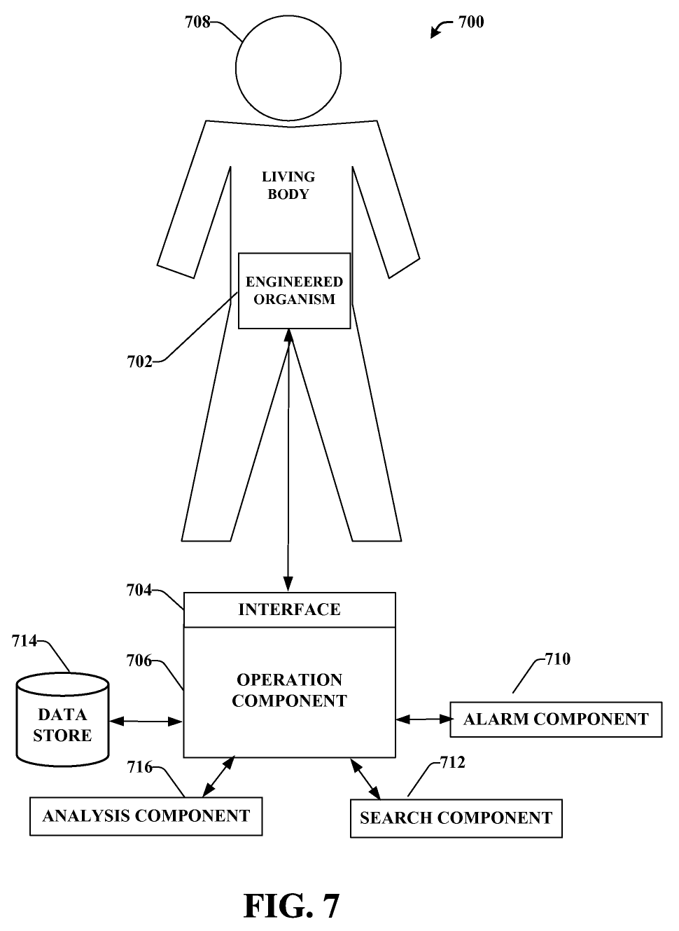

FIG. 7 illustrates a system 700 that facilitates implementing engineered organism within a living body 708 providing alarms, data logging/searching, event logging, and/or querying. The system 700 can include an engineered organism 702 that provides monitoring, detection, logging, and/or data collection within a living body 708. The collected encoded data can be utilized by an operation component 706, wherein the encoded data can be decoded using a data standard for biomaterials and implemented for analysis, computations, determinations, and/or any other manipulations based upon the collected data.

The operation component 706 can utilize an alarm component 710 that can provide alarms and/or warnings associated with encoded data generated by the engineered organism 702 or decoded data generated by operation component 706 based at least in part upon the health severity of the measurements, detections, and/or data collections via the engineered organism 702 and/or historical/nominal data. For example, the alarm component 710 can implement an alarm or unambiguous warning such as, warning lights, pop-up screens, blinking data display items, graphical items, email, text, cellular communication, web site activity, etc. when a particular condition is detected or measured beyond a specific threshold. It is to be appreciated that the status and/or alarms can be stored in the data store 714 (described previously), a “black box” recorder, etc. Additionally, the alarm component can signal a condition that may occur in the future to permit action to be taken before a condition occurs or before actions causing a condition are performed.

The operation component 706 can further utilize a search component 712 that allows querying of the system 700. In particular, the search component 712 can provide querying of any encoded data generated by the engineered organism 702, decoded data generated by the operation component 706, stored data, logged data, system data, conditions of the living body 708, analytical data, historical data and/or any other data related to the system 700. For instance, the search component 712 can be utilized to discover data related to a particular condition and circumstances present when the condition was measured/detected. It is to be appreciated that although the search component 712 is illustrated as a stand-alone component, the search component 712 can be incorporated into the operation component 706. It is to be appreciated that the system components can be distributed and remote from the living body 708 and remote from each other such as accessible via Internet, Ethernet, etc.

The system 700 can include an analysis component 716, which can analyze the significance of encoded data generated by the engineered organism 702, decoded data generated by the operation component 706, and prescribed suitable countermeasures to conditions detected within the living body 708.

Data generated by symbiotic agents may be collected and analyzed, providing powerful information to devise new and alternative treatments and interventions for diseases, deficiencies, preventions, and so forth.

In one embodiment, a genetically engineered Lactobacillus acidophilus is provided to a patient with diabetes through ingestion of yogurt containing the engineered bacteria. The engineered Lactobacillus acidophilus colonizes the digestive tract, and is programmed to monitor glucose and/or insulin levels. After a suitable period of time to collect information of glucose levels and/or insulin levels elapses, a tablet containing a chemical that acts as a stimulus to the engineered Lactobacillus acidophilus

and signals the engineered Lactobacillus acidophilus to release biomaterial containing a logfile of the glucose levels and/or insulin levels. The tablet can also be taken on regular time interval, such as every week, to provide information on glucose levels and/or insulin levels over an extended period of time. The biomaterial is obtained by collecting urine, blood, or some other substance from the patient and subjected to analysis whereby the logfile is read. Another tablet containing a chemical that acts as a stimulus to the engineered Lactobacillus acidophilus and signals the Lactobacillus acidophilus to induce apoptosis.

In another embodiment, a patient drinks a culture of genetically modified Candida albicans. The yeast colonize the pancreas. Sometime later, the patient is given a first biologically inert small molecule that stimulates the genetically modified Candida albicans to start logfile production. Several hours later, the patient is given a second biologically inert small molecule that stimulates the genetically modified Candida albicans to record insulin production. A logfile entry is recorded in the form of a binary sequence on the DNA logfile, followed by a further binary sequence acting as a spacer. Several hours later, the patient is given a third biologically inert small molecule that stimulates the genetically modified Candida albicans to cumulatively record insulin molecules it encounters. The stimulus decays after a set time. A logfile entry encoding insulin molecule numbers is recorded in the form of a binary sequence on the DNA logfile, followed by a further binary sequence acting as a spacer. Several hours later, the patient is given a fourth biologically inert small molecule that causes the bacterium to deactivate the logging process, terminate the binary DNA logfile and tag it with a protein so the cellular machinery exports it. The patient is then given a fifth biologically inert small molecule that induces apoptosis in the genetically modified Candida albicans. Logfile molecules are collected from patient blood and read using any of the sequencing technologies, building up a picture of pancreatic function far beyond resolution using conventional techniques.

In one embodiment, the engineered organism is a friendly bacteria transformed with a gene that expresses a peptide containing alanine and valine in accordance with a data standard. The engineered organism contains a glucose receptor for sensing the presence/amount of glucose. The data standard includes first amino acid sequence code for blood glucose levels below 4 mmol/l, a second amino acid sequence code for blood glucose levels from 4 mmol/l to 6 mmol/l, and a third amino acid sequence code for blood glucose levels exceeding 6 mmol/l. The engineered organism is administered to a human subject afflicted with diabetes (type 1 or type 2). Once in the human subject, the engineered organism senses its environment, constantly determining the blood glucose level and logging the amount of blood glucose in a peptide/protein expressed by the engineered organism. A blood sample is taken from the human subject, and the expressed peptide/protein containing only alanine and valine is isolated and sequenced. The amino acid sequence is interpreted/read in light of the data standard which provides the blood glucose level over a period of time. The blood glucose level can be mapped to the food intake record, enabling a detailed study of the human subject's insulin production in response to food intake.

In a related embodiment, a first engineered organism is a friendly bacteria transformed with a gene that expresses a peptide containing alanine and valine in accordance with a data standard and causes expression of a signal compound when blood glucose level exceeds a predetermined threshold amount and a second engineered organism is a friendly bacteria transformed with a gene that expresses insulin or proinsulin when a signal compound is detected. The second engineered organism contains a receptor for the signal compound, and when the signal compound binds to the receptor, expression of insulin or proinsulin is activated. The data standard includes first amino acid sequence code for blood glucose levels below 4 mmol/l, a second amino acid sequence code for blood glucose levels from 4 mmol/l to 6 mmol/l, and a third amino acid sequence code for blood glucose levels exceeding 6 mmol/l. The data standard also includes a fourth amino acid sequence code for the release of the signal compound, which occurs when the blood glucose levels exceed 6.1 mmol/l.

The engineered organisms are administered to a human subject afflicted with diabetes. Once in the human subject, the first engineered organism senses its environment, constantly determining the blood glucose level and logging the amount of blood glucose in a peptide/protein expressed by the engineered organism and constantly determining whether or not the blood glucose level exceeds or does not exceed the predetermined threshold amount. When the blood glucose level does not meet the predetermined threshold amount, the first engineered organism expresses no signal compound. When the blood glucose level exceeds the predetermined threshold amount, the first engineered organism expresses a signal compound. The presence of the signal compound induces the second engineered organism to express insulin or proinsulin until the blood glucose levels are below 6.1 mmol/l.

In yet another embodiment, the engineered organism is L. acidophilus transformed with a gene that encodes for the expression of a protein containing only glycine and glutamine in accordance with a data standard. L. acidophilus is selected for transformation due to its natural presence in the colon. The engineered organism contains a receptor for hemoglobin for detecting the presence of red blood cells, and/or other components not normally present in the colon. The data standard includes first amino acid sequence code for hemoglobin levels exceeding a predetermined threshold, a second amino acid sequence code for another component not normally present in the colon, and a third amino acid sequence code for yet another component not normally present in the colon. The engineered organism is administered to a human subject afflicted with polyps in the colon or at risk for colon cancer. Once in the human subject, the engineered organism senses its environment, constantly determining the hemoglobin level (and levels of other components not normally present in the colon) and logging the amount of hemoglobin/blood in a peptide/protein expressed by L. acidophilus. A blood sample is taken from the human subject, and the peptide/protein expressed by L. acidophilus containing only residues of glycine and glutamine is sequenced. The blood glucose level can be mapped to the food intake record, enabling a detailed study of the human subject's insulin production in response to food intake. The signal compound is readily detectable in feces excreted from the human subject. By isolating and sequencing the expressed peptide/protein containing only glycine and glutamine, and comparing the amino acid sequence in light of the data standard, the level of rectal bleeding is tracked. Consequently, one is in fact monitoring for a symptom of colon cancer; namely, rectal bleeding. Since low levels of rectal bleeding are often difficult to detect, especially for those with untrained eyes, the engineered organism provides an effective and alternative opportunity to detect colon cancer.

In still yet another embodiment, the engineered organism is a liver fluke genetically engineered to secrete a chemotherapeutic compound when alpha-fetoprotein is present or sensed. The liver fluke is also transformed with a gene that encodes for the expression of a protein containing only proline and serine in accordance with a data standard. The liver fluke contains an alpha-fetoprotein receptor for detecting the presence of alpha-fetoprotein (a generally accepted marker of a liver tumor). The data standard includes first amino acid sequence code for alpha-fetoprotein levels exceeding a predetermined threshold, a second amino acid sequence code for the secretion of the chemotherapeutic compound, and a third and fourth amino acid sequence codes for the presence and absence of alpha-fetoprotein, respectively. The liver fluke is genetically engineered so that the binding of alpha-fetoprotein to the alpha-fetoprotein receptor activates secretion of the chemotherapeutic compound, which is effective in treating liver cancer. Once in a human subject, the liver fluke naturally migrates to the liver, senses for alpha-fetoprotein, and produces a chemotherapeutic compound when alpha-fetoprotein is sensed. The liver fluke expresses a protein containing only proline and serine that reports on presence/absence of alpha-fetoprotein, whether alpha-fetoprotein levels exceed a predetermined threshold, and whether a chemotherapeutic compound is employed to treat liver cancer. The expressed protein is isolated, sequenced, and read using the data standard.

It is to be appreciated and understood that directions in synthetic biology are enabling increasingly flexible and reusable specifications of systems based on inventories of well-characterizable components and assemblies such as channels, timers, switches, etc. Moreover, introduced biological systems can employ components that record and/or remember multiple states over time, as encoded in a single and/or multiple molecules.

The techniques, systems, and methods described herein further include engineering colonies or, more generally, systems of different types of bacteria and/or parasites. The properties of such larger systems provide designers more flexibility to sense, produce, or intervene. In general, the rich control and generative machinery of symbiotes—as well as, where necessary pathogens and parasites are exploited to perform the desired tasks that improve the overall health of living bodies.

As utilized herein, terms “component,” “system,” “interface,” “device,” “generator,” “collector,” and the like are intended to refer to a computer-related entity, either hardware, software (e.g., in execution), and/or firmware. For example, a component can be a process running on a processor, a processor, an object, an executable, a program, a function, a library, a subroutine, and/or a computer or a combination of software and hardware. By way of illustration, both an application running on a server and the server can be a component. One or more components can reside within a process and a component can be localized on one computer and/or distributed between two or more computers.

Furthermore, the claimed subject matter may be implemented as a method, apparatus, or article of manufacture using standard programming and/or engineering techniques to produce software, firmware, hardware, or any combination thereof to control a computer to implement the disclosed subject matter. The term “article of manufacture” as used herein is intended to encompass a computer program accessible from any computer-readable device, carrier, or media. For example, computer readable media can include but are not limited to magnetic storage devices (e.g., hard disk, floppy disk, magnetic strips . . . ), optical disks (e.g., compact disk (CD), digital versatile disk (DVD) . . . ), smart cards, and flash memory devices (e.g., card, stick, key drive . . . ). Additionally it should be appreciated that a carrier wave can be employed to carry computer-readable electronic data such as those used in transmitting and receiving electronic mail or in accessing a network such as the Internet or a local area network (LAN). Of course, those skilled in the art will recognize many modifications may be made to this configuration without departing from the scope or spirit of the claimed subject matter. Moreover, the word “exemplary” is used herein to mean serving as an example, instance, or illustration. Any aspect or design described herein as “exemplary” is not necessarily to be construed as preferred or advantageous over other aspects or designs.

While the claimed subject matter has been described above in the general context of computer-executable instructions of a computer program that runs on a local computer and/or remote computer, those skilled in the art will recognize that the subject innovation also may be implemented in combination with other program modules. Generally, program modules include routines, programs, components, data structures, etc., that perform particular tasks and/or implement particular abstract data types.

Moreover, those skilled in the art will appreciate that the inventive methods may be practiced with other computer system configurations, including single-processor or multi-processor computer systems, minicomputers, mainframe computers, as well as personal computers, hand-held computing devices, microprocessor-based and/or programmable consumer electronics, and the like, each of which may operatively communicate with one or more associated devices. The illustrated aspects of the claimed subject matter may also be practiced in distributed computing environments where certain tasks are performed by remote processing devices that are linked through a communications network. However, some, if not all, aspects of the subject innovation may be practiced on stand-alone computers. In a distributed computing environment, program modules may be located in local and/or remote memory storage devices.

What has been described above includes examples of the subject innovation. It is, of course, not possible to describe every conceivable combination of components or methodologies for purposes of describing the claimed subject matter, but one of ordinary skill in the art may recognize that many further combinations and permutations of the subject innovation are possible. Accordingly, the claimed subject matter is intended to embrace all such alterations, modifications, and variations that fall within the spirit and scope of the appended claims.

In particular and in regard to the various functions performed by the above described components, devices, circuits, systems and the like, the terms (including a reference to a “means”) used to describe such components are intended to correspond, unless otherwise indicated, to any component which performs the specified function of the described component (e.g., a functional equivalent), even though not structurally equivalent to the disclosed structure, which performs the function in the herein illustrated exemplary aspects of the claimed subject matter. In this regard, it will also be recognized that the innovation includes a system as well as a computer-readable medium having computer-executable instructions for performing the acts and/or events of the various methods of the claimed subject matter.

There are multiple ways of implementing the present innovation, e.g., an appropriate API, tool kit, driver code, operating system, control, standalone or downloadable software object, etc. which enables applications and services to use the techniques of the invention. The claimed subject matter contemplates the use from the standpoint of an API (or other software object), as well as from a software or hardware object that operates according to the techniques in accordance with the invention. Thus, various implementations of the innovation described herein may have aspects that are wholly in hardware, partly in hardware and partly in software, as well as in software.

The aforementioned systems have been described with respect to interaction between several components. It can be appreciated that such systems and components can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it should be noted that one or more components may be combined into a single component providing aggregate functionality or divided into several separate sub-components, and any one or more middle layers, such as a management layer, may be provided to communicatively couple to such sub-components in order to provide integrated functionality. Any components described herein may also interact with one or more other components not specifically described herein but generally known by those of skill in the art.

In addition, while a particular feature of the subject innovation may have been disclosed with respect to only one of several implementations, such feature may be combined with one or more other features of the other implementations as may be desired and advantageous for any given or particular application. Furthermore, to the extent that the terms “includes,” “including,” “has,” “contains,” variants thereof, and other similar words are used in either the detailed description or the claims, these terms are intended to be inclusive in a manner similar to the term “comprising” as an open transition word without precluding any additional or other elements.

What is claimed is:

1. A system that facilitates monitoring a condition of a living body, comprising:
an engineered organism that produces a biomaterial containing encoded information;
an interface component that facilitates receipt of or delivery of the biomaterial; and
an operation component that analyzes the biomaterial using a data standard, the data standard associating each of a set of stimuli with a corresponding each of a set of biomaterial subunit sequences.

2. The system of claim 1, the data standard comprises binary code.

3. The system of claim 1, the stimuli comprise at least one of vitamin levels/production, mineral levels, amino acid levels/production/reduction, fatty acid levels/production, hormone levels/production, insulin levels/production, hemoglobin levels/production, glucose levels, alcohol levels, infections, parasitic infections, genetic disorders, cancer, cancer producing substances, presence of toxins, abnormal physical states, allergen levels, drug metabolite levels, immune response compounds levels/production, or melanin levels/production.

4. The system of claim 1, the living body comprising a human being.

5. The system of claim 1, the biomaterial is one of a nucleotide sequence or an amino acid sequence.

6. The system of claim 1, the engineered organism comprising a genetically engineered bacterium.

7. The system of claim 1, the engineered organism comprising a genetically engineered parasite.

8. A method of logging events in a living body, comprising:
introducing an engineered organism into a living body, the engineered organism encodes events in a biomaterial;
collecting the biomaterial generated by the engineered organism, the biomaterial comprising encoded data indicative of an event in the living body; and
using a data standard to correlate the encoded data with the event.

9. The method of claim 8 further comprising sequencing the biomaterial generated by the engineered organism before using the data standard.

10. The method of claim 8, the data encoded in the biomaterial according to binary code.

11. The method of claim 8, the data encoded in the biomaterial according to ASCII code.

12. The method of claim 8, collecting the biomaterial comprising collecting one of blood, urine, saliva, feces, mucus, or hair from the living body.

13. The method of claim 8, the engineered organism encodes events in a polymeric biomaterial comprising two different subunits.

14. An event logging system that obtains data concerning a living body, comprising:
an engineered organism that produces a biomaterial containing encoded information in response to a stimulus; and
a data standard for correlating each of a set of stimuli with a unique biomaterial sequence code.

15. The event logging system of claim 14, the biomaterial is one of a nucleotide sequence or an amino acid sequence.

16. The event logging system of claim 14, the data standard comprises binary code.

17. The event logging system of claim 14, the biomaterial is a polymer comprising two different subunits.

18. The event logging system of claim 14, the encoded information comprising an event log.

19. The event logging system of claim 14, the engineered organism comprising a genetically engineered bacterium.

20. The event logging system of claim 14, the stimuli comprise at least one of vitamin levels/production, mineral levels, amino acid levels/production/reduction, fatty acid levels/production, hormone levels/production, insulin levels/production, hemoglobin levels/production, glucose levels, alcohol levels, infections, parasitic infections, genetic disorders, cancer, cancer producing substances, presence of toxins, abnormal physical states, allergen levels, drug metabolite levels, immune response compounds levels/production, or melanin levels/production.
